(19) United States
(12) Patent Application Publication
(10) Pub. No.: US 2011/0251672 A1
(43) Pub. Date: Oct. 13, 2011

(54) ASPECT RATIO FOR STENT STRUT DESIGN

(75) Inventor: CHAD J. ABUNASSAR, San Francisco, CA (US)

(73) Assignee: ABBOTT CARDIOVASCULAR SYSTEMS INC., Santa Clara, CA (US)

(21) Appl. No.: 12/758,197

(22) Filed: Apr. 12, 2010

(51) Int. Cl. A61F 2/82

(52) U.S. Cl. 623/1.16; 623/1.15; 623/1.42

(57) ABSTRACT

An intravascular stent is provided to be implanted in coronary arteries and other body lumens. The stent includes stent struts that have a transverse cross-section that is substantially rectangular and an aspect ratio in the range of 0.9 to 1.0 to 1.4 to 1.0. This range of aspect ratios for the stent struts minimizes out of plane twisting of the struts or projecting edges of the struts when the stent is expanded from a compressed diameter to an expanded diameter in a coronary artery.

### BACKGROUND

The invention relates to expandable stents which are adapted to be implanted into a patient's body lumen such as a coronary artery, in order to maintain the patency thereof. Stents are useful in the treatment of atherosclerotic stenosis in the coronary arteries, and other vessels in the body.

Stents are generally tubular-shaped devices which function to hold open a segment of a blood vessel or coronary artery, or other anatomical lumen. They also are useful to support and hold back a dissected arterial lining which can occlude the fluid passageway therethrough. The delivery and deployment of stents in the coronary arteries are well known in the art and various types of catheters are used, along with guide wires, to position and implant a stent in an artery.

Stents typically are formed from thin-walled metal tubing that is laser cut to form a pattern of stent struts in the tubing wall. The stent struts will typically have a generally rectangular cross-section when formed by the laser cutting. One of the difficulties encountered in forming stents having struts with a rectangular cross-section is that the ability to uniformly compress the stent onto the balloon portion of a catheter and expand the stent for implanting into a coronary vessel is not uniform and results in twisting or projecting edges. For example, in U.S. Pat. No. 5,514,154, which is incorporated herein by reference, the struts have an aspect ratio resulting in projecting edges when the stent is expanded and implanted in a coronary artery.

What has been needed and heretofore unavailable is a strut aspect ratio that provides a stent that can be uniformly compressed and expanded without developing out-of-plane twisting of the stent struts or projecting edges of the struts.

### SUMMARY OF THE INVENTION

The present invention is directed to a stent formed from an elongated tubular member having struts that form a stent pattern. The struts have a transverse cross-section that is generally rectangular, while the edges may be electro-polished so that the edges are rounded while still maintaining the generally rectangular cross-sectional shape. The struts have an aspect ratio from about 0.9 to 1.0 to about 1.4 to 1.0 which is the optimum aspect ratio in order to compress the stent onto the balloon portion of a catheter and expand the stent into an artery without the struts twisting out of plane or forming projecting edges.

In one embodiment, the stent struts have a width ranging from 80 μm (0.00315 inch) to 97 (0.0038 inch) μm and the struts have a radial thickness (height) ranging from 56 μm (0.0022 inch) to about 81 μm (0.0032 inch). When forming a stent pattern using a laser, the strut width and strut radial thickness can be selected from any of these dimensions as long as the aspect ratio falls within the range of about 0.9 to 1.0 to about 1.4 to 1.0. Stent struts having these aspect ratios will have minimal out-of-plane twisting and reduce the likelihood that portions of the stent will project radially outwardly when the stent is expanded into an arterial wall.

In one embodiment, the stent as previously described has linear strut segments and curved strut segments that have different aspect ratios. The linear strut segments may have an aspect ratio that is either more or less than the aspect ratio of the curved strut segments depending upon a particular application.

Other features and advantages of the present invention will become more apparent from the following detailed description of the invention when taken in conjunction with the accompanying exemplary drawings.

### BRIEF DESCRIPTION OF THE DRAWINGS

FIG. 1 is an enlarged, partial perspective view of a prior art stent showing a portion of a strut segment projecting radially outwardly after expansion.

FIG. 2 is an elevational view depicting a stent uniformly compressed onto a balloon portion of a catheter and having an aspect ratio of from 0.9 to 1.0 to 1.4 to 1.0.

FIG. 3 is an elevational view depicting an expanded stent on a balloon and having an aspect ratio that minimizes out-of-plane twisting of the struts and minimizes projecting edges.

FIG. 4 is an elevational view depicting an expanded stent having an aspect ratio that minimized out-of-plane twisting of the struts and minimizes projecting edges.

FIG. 5 is a transverse cross-sectional view of a strut of the stent of the present invention having a substantially rectangular shape.

### DETAILED DESCRIPTION OF THE PREFERRED EMBODIMENTS

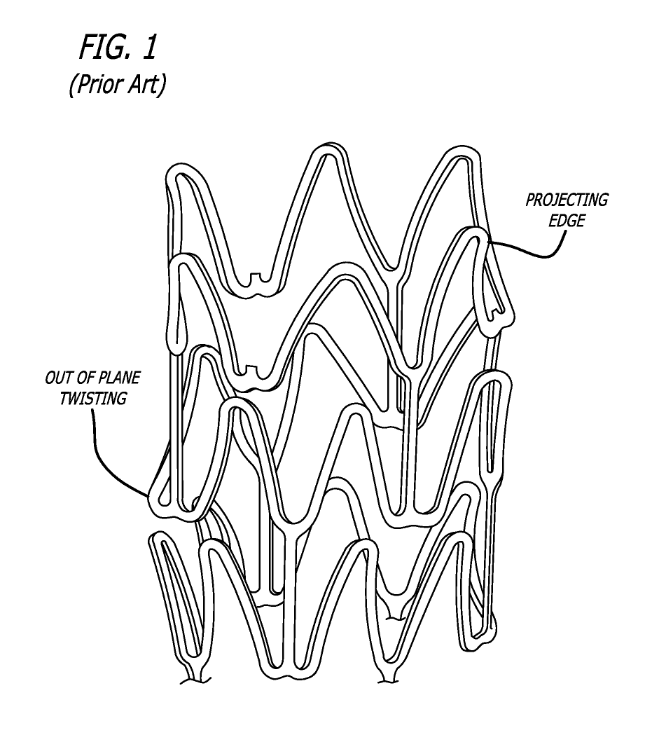

Intravascular stents are generally formed by laser cutting a pattern in a thin walled tube and then etching and/or electropolishing the laser cut stent. This typically produces a stent strut that has a transverse cross-section that is generally square or rectangular with somewhat rounded corners. First generation stent strut cross-sections were generally square in nature, however, clinical trials have shown that thinner struts (less radial thickness) perform better with respect to limiting the formation of restenosis. This conclusion is attributed to the observation that radially thinner stent struts drive a reduction in arterial injury and thus provide less disruption of local hemodynamics when compared to radially thicker stent struts. Even though thin stent struts provide these clinical benefits, they also must be made stronger and/or stiffer to provided sufficient radial strength and stiffness in order to properly scaffold a target lesion or arterial wall. These thin struts therefore exhibit higher aspect ratios (strut width strut height) greater than one-to-one (square) to provide sufficient bending stiffness to prevent the stent struts from closing due to strut bending loads. In designing a strut with a high aspect ratio, however, the typical strut torsional resistance is relatively low compared to a square (symmetric) cross-section. When torsional resistance is low compared to the bending resistance of a stent strut (which is the case for high aspect ratio struts), the stent strut may twist slightly out of plane when undergoing initial elastic deformation. As shown in FIG. 1, this effect is more severe under large plastic deformation due to compressing or crimping the stent onto the balloon portion of a catheter or during stent expansion into an artery. This localized twisting leads to irregular out-of-plane strut deformations, which are associated with local strut fracture, increased local arterial injury, poor local scaffolding and subsequent plaque prolapse, poor drug delivery (for stents having a drug coating), and an incomplete strut apposition with a potential for associated thrombosis.

In keeping with the present invention, a stent strut cross-section is provided to ensure optimal stent crimping and stent expansion. More specifically, the present invention design provides uniform stent expansion that provides the following potential benefits: maximized radial strength and thickness; uniform strut apposition; reliable side-branch access; reduced local vessel injury; improved uniformity of drug delivery; and improved circular expansion. Further, the strut cross-section of the present invention is associated with an improved crimp profile, uniformity in crimping, and improved stent retention on the balloon portion of a catheter.

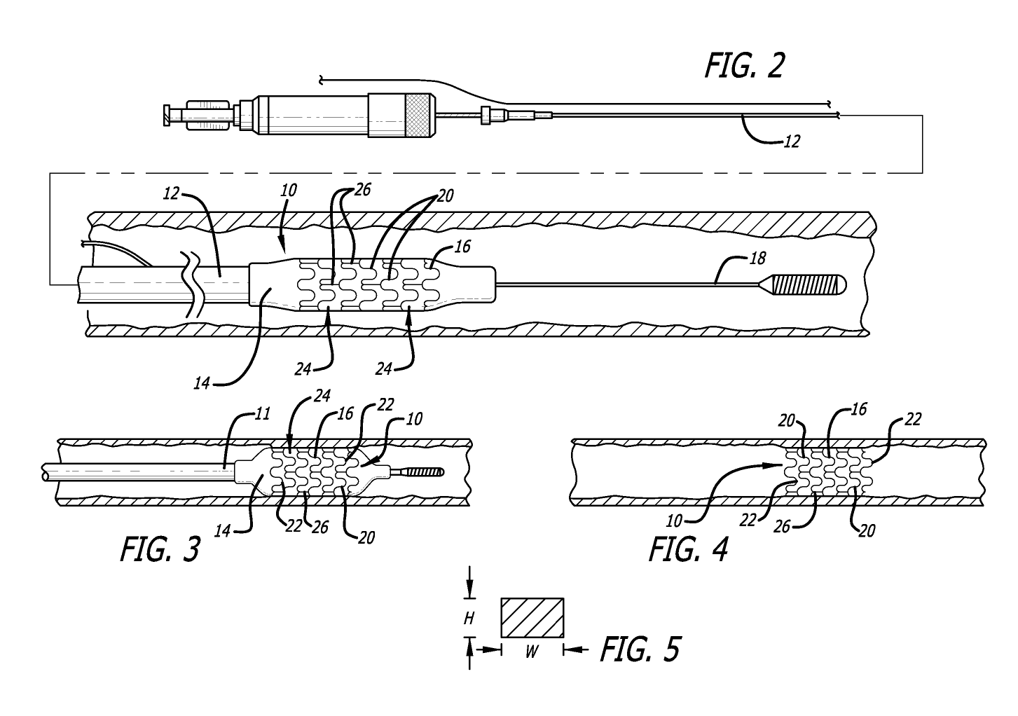

In keeping with the invention, and referring to FIGS. 2-4, a stent delivery assembly 10 includes a rapid exchange catheter 12 which has an expandable balloon 14 at its distal end. An intravascular stent 16 is mounted on the balloon and compressed firmly onto the balloon in a known manner. A guide wire 18 extends through at least a portion of the distal end of catheter 12 so that the catheter can slide along the guide wire and into the coronary arteries, also in a known manner. Once the catheter 12 positions the stent in a coronary artery, the stent is expanded radially outwardly by expanding the balloon portion of the catheter as shown in FIGS. 3 and 4, so that the stent is pressed into the coronary artery or it is permanently implanted to hold open the artery and prevent recoil.

In one embodiment of the present invention, as shown in FIGS. 3 and 4, stent 16 has multiple linear stent struts 20 (sometimes referred to as bar arms) interconnecting with curved stent struts 22. A stent pattern having linear stent struts and curved stent struts is generally formed from a tubular member by laser cutting in a known manner which is well known in the art. The stent struts form cylindrical rings 24 that extend circumferentially and can be compressed radially inwardly onto the balloon 14, or expanded radially outwardly when the balloon is expanded in order to implant the stent in a coronary artery. The rings are interconnected with links 26, which include the linear stent struts 20.

In one aspect of the invention, as shown in FIG. 5, the linear stent struts 20 and the curved stent struts 22 have a transverse cross-section that is substantially rectangular. The struts have a width W and a height H where the ratio of the width divided by the height is the aspect ratio of the strut. With respect to the present invention, an optimum aspect ratio can range from 0.9 to 1.0 to about 1.4 to 1.0. More preferably, an optimum aspect ratio range is 1.2 to 1.0 to 1.4 to 1.0. More preferably, an optimum aspect ratio is about 1.4 to 1.0. In these aspect ratio ranges, when the stent is compressed onto the balloon, and expanded from the compressed diameter to the expanded diameter in a coronary artery, the struts will not twist out of plane or project radially outwardly even though the metal plastically deforms. Any rectangular or square shape exceeding this aspect ratio range will result in the struts twisting out of plane or the curved portions projecting radially outwardly when the stent is expanded and plastically deformed. In order to fall within the desired aspect ratio range, the stent struts 20 and 22 have a width W ranging from 80 μm (0.00315 inch) to 97 μm (0.0038 inch) and a radial thickness or height H ranging from 56 μm (0.022 inch) to 81 μm (0.0032 inch). Any combination of the width and height in the disclosed ranges that provide an aspect ratio in the range of about 0.9 to 1.0 to about 1.4 to 1.0 will provide a strut that does not appreciably twist out of plane or project radially outwardly. Further, struts having these dimensions will have maximum radial strength and stiffness and will have uniform strut expansion, and improve the uniformity of drug delivery if they are drug coated. Further, since many stents currently have rings 24 that expand radially outwardly, the disclosed width and height ranges also provide a stent that provides a near circular expansion.

While the invention has been illustrated and described herein in terms of its use as an intravascular stent, it will be apparent to those skilled in the art that the stent can be used in other instances such as to expand body lumens and other vessels in addition to coronary arteries. Other modifications and improvements can be made without departing from the scope of the invention.

What is claimed:

1. A stent, comprising:
an elongated tubular member having a plurality of struts forming a stent pattern;
the struts having a transverse cross-section that is generally rectangular;
the elongated tubular member having a first compressed diameter and a second expanded diameter; and
the struts having an aspect ratio in the range of about 0.9 to 1.0 to about 1.4 to 1.0 so that as the stent is expanded from the compressed diameter to the expanded diameter, the struts do not twist out of plane or project radially outwardly.

2. The stent of claim 1, wherein the struts have a width ranging from 80 μm (0.00315 inch) to 97 μm (0.0038 inch) and the struts have a radial thickness ranging from 56 μm (0.0022 inch) to 81 μm (0.0032 inch).

3. The stent of claim 1, wherein the struts have linear segments and curved segments.

4. The stent of claim 3, wherein the linear segments and the curved segments have substantially the same aspect ratio.

5. The stent of claim 3, wherein the linear segments and the curved segments have different aspect ratios.

6. The stent of claim 3, wherein the linear segments have an aspect ratio that is less than the aspect ratio of the curved segments.

7. The stent of claim 3, wherein the linear segments have an aspect ratio that is greater than the aspect ratio of curved segments.

8. The stent of claim 1, wherein the stent struts are formed from a metallic material.

9. The stent of claim 1, wherein the stent struts are formed from a non-metallic material.

10. The stent of claim 9, wherein the non-metallic material includes a polymer material.

11. The stent of claim 8, wherein the metallic material is plastically deformable.

12. The stent of claim 1, wherein the struts have an aspect ratio of no greater than 1.4 to 1.0.

13. The stent of claim 1, wherein the struts have a therapeutic drug coating.

14. A stent, comprising:
an elongated tubular member having plurality of linear strut segments and curved strut segments;
the struts having a transverse cross-section that is generally rectangular;
the elongated tubular member having a first compressed diameter and a second expanded diameter; and
at least some of the curved strut segments have an aspect ratio no greater than 1.4 to 1.0 so that as the stent is expanded from the compressed diameter to the expanded diameter, the at least some of the curved strut segments do not twist out of plane or project radially outwardly.

15. The stent of claim 14, wherein the struts have a width ranging from 80 μm (0.00315 inch) to 97 μm (0.0038 inch) and the struts have a radial thickness ranging from 56 μm (0.0022 inch) to 81 μm (0.0032 inch).

16. The stent of claim 14, wherein the linear segments and the curved segments have substantially the same aspect ratio.

17. The stent of claim 14, wherein the linear segments and the curved segments have different aspect ratios.

18. The stent of claim 14, wherein the linear segments have an aspect ratio that is less than the aspect ratio of the curved segments.

19. The stent of claim 14, wherein the linear segments have an aspect ratio that is greater than the aspect ratio of curved segments.

20. The stent of claim 14, wherein the stent struts are formed from a metallic material.

21. The stent of claim 14, wherein the stent struts are formed from a non-metallic material.

22. The stent of claim 21, wherein the non-metallic material includes a polymer material.

23. The stent of claim 20, wherein the metallic material is plastically deformable.

24. The stent of claim 14, wherein the struts have an aspect ratio of no greater than 1.4 to 1.0.

25. A stent, comprising:
an elongated tubular member having a plurality of struts forming a stent pattern;
the struts having a transverse cross-section that is generally rectangular;
the elongated tubular member having a first compressed diameter and a second expanded diameter; and
the struts having an aspect ratio in the range of about 1.2 to 1.0 to about 1.4 to 1.0 so that as the stent is expanded from the compressed diameter to the expanded diameter, the struts do not twist out of plane or project radially outwardly.

26. The stent of claim 25, wherein the struts have a width ranging from 80 μm (0.00315 inch) to 97 μm (0.0038 inch) and the struts have a radial thickness ranging from 56 μm (0.0022 inch) to 81 μm (0.0032 inch).

27. The stent of claim 25, wherein the struts have linear segments and curved segments.

28. The stent of claim 27, wherein the linear segments and the curved segments have substantially the same aspect ratio.

29. The stent of claim 27, wherein the linear segments and the curved segments have different aspect ratios.

30. The stent of claim 27, wherein the linear segments have an aspect ratio that is less than the aspect ratio of the curved segments.

31. The stent of claim 27, wherein the linear segments have an aspect ratio that is greater than the aspect ratio of curved segments.
